(19) United States
(12) Patent Application Publication
(10) Pub. No.: US 2025/0027035 A1
(43) Pub. Date: Jan. 23, 2025

(54) HEAVY METAL-ANTIBIOTIC MULTIPLE RESISTANT BACTERIA AND ITS APPLICATION

(71) Applicant: SHANDONG UNIVERSITY, Weihai City (CN)

(72) Inventors: Mengqi YE, Weihai City (CN); Zongjun DU, Weihai City (CN); Yuqi YE, Weihai City (CN); Xinyun TAN, Weihai City (CN); Hansheng ZHU, Weihai City (CN); Zhipeng HAO, Weihai City (CN)

(73) Assignee: SHANDONG UNIVERSITY, Weihai City (CN)

(21) Appl. No.: 18/676,524

(22) Filed: May 29, 2024

(30) Foreign Application Priority Data
Jul. 18, 2023 (CN) 2023108823877

(51) Int. Cl. C12N 1/20; B09C 1/10; C02F 3/34; C02F 101/22; C02F 103/34; C12R 1/01

(52) U.S. Cl. CPC C12N 1/205; B09C 1/10; C02F 3/34; C02F 2101/22; C02F 2103/343; C12R 2001/01

(57) ABSTRACT

The invention relates to a heavy metal-antibiotic multiple resistant bacteria and its application, which belongs to the field of microbial remediation technology. The heavy metal-antibiotic multiple resistant bacteria described in this paper is Rossellomorea oryzaecorticis ZC255, which is preserved at China General Microbiological Culture Collection Center on Jun. 12, 2023, the biological preservation number is CGMCC No. 27617. This strain not only has high activity, simple culture method and strong adaptability, but also can survive in solid medium with high concentration of heavy metals and antibiotics. It also has excellent metal tolerance; it can effectively degrade a variety of antibiotics and can be applied to the remediation of heavy metal-contaminated or antibiotic-contaminated or heavy metal and antibiotic-contaminated water or soil. In addition, it has the advantages of large adsorption capacity, low cost, high efficiency, good effect and no secondary pollution.

### TECHNICAL FIELD

The invention belongs to the field of microbial remediation technology, and specifically relates to a heavy metal-antibiotic multiple resistant bacteria and its application.

### BACKGROUND ART

The disclosure of the information of the technical part is only intended to increase the understanding of the general background of the invention, and is not necessarily regarded as an acknowledgement or any indication that the information has become a prior technology known to the general technicians in the field.

Bioremediation is an efficient, economical and more sustainable treatment method for contaminated medium, which uses the removal ability and adsorption ability or accumulation ability of microorganisms to control environmental pollution. Bacteria can adsorb, accumulate, and convert most heavy metals and antibiotics into harmless forms. Heavy metals and antibiotic resistant bacteria have important potential in bioremediation, and their resistance and accumulation ability ensure that they can be used for bioremediation of the environment. Compared with conventional traditional technology, the use of resistant bacteria for environmental bioremediation has the characteristics of large adsorption capacity, low cost, high efficiency, and no secondary pollution. However, the current heavy metals and antibiotic resistant bacteria generally have problems such as poor removal of heavy metals and antibiotics.

At present, the germ plasm resources of resistant bacteria that can degrade heavy metals and antibiotics are scarce. It is necessary to provide more and more multi-functional resistant bacteria for the removal of heavy metals or antibiotics or the pollutants of heavy metals and antibiotics in water and soil, which can well repair the environment polluted by heavy metals and antibiotics.

### SUMMARY

The technical problem to be solved by the invention is to overcome the above defects and deficiencies, and provide a heavy metal-antibiotic multiple resistant bacteria and its application.

The first purpose of the invention is to provide a heavy metal-antibiotic multiple resistant bacteria.

The second purpose of the invention is to provide a bacteria agent containing the heavy metal-antibiotic multiple resistant bacteria.

The third purpose of the invention is to provide the application of the heavy metal-antibiotic multiple resistant bacteria or bacteria agents in the removal of antibiotics and/or in the preparation of antibiotic degradation agents.

The fourth purpose of the invention is to provide the application of the heavy metal-antibiotic multiple resistant bacteria or bacteria agents in the remediation of antibiotic-contaminated environment or heavy metal-contaminated environment or heavy metal and antibiotic-contaminated environment.

The fifth purpose of the invention is to provide a method for degrading antibiotics.

The above purpose of the invention is realized by the following technical scheme:

In the first aspect of the invention, a heavy metal-antibiotic multiple resistant bacteria is provided, the heavy metal-antibiotic multiple resistant bacteria is Rossellomorea oryzaecorticis ZC255, the strain has been preserved on Jun. 12,2023 at the China General Microbiological Culture Collection Management Center, the address is: No. 3, Courtyard No. 1, Beichen West Road, Chaoyang District, Beijing, the biological preservation number is CGMCC No. 27617. The bacterial strain is isolated and purified from marine sediments of the sea area where is near Shandong Weihai Xiaoshi Island, it is a bacteria strain with heavy metal-antibiotic resistance ability. The strain is identified as Rossellomorea oryzaecorticis by morphological characteristics and 16 s rRNA gene phylogenetic analysis, and the strain is Gram-positive bacteria, the color of colony is orange-red, the shape of strain is spherical, the growth temperature range is 15˜45° C., the growth pH range is 6˜9, and the NaCl concentration range is 0˜18%. The results of bacterial enzyme activity assay shows that the strain has oxidase, lipase, caseinase, amylase, cellulase and other activities.

The research of the invention shows that the Rossellomorea oryzaecorticis ZC255 strain has good tolerance and adsorption ability to Cd²⁺, Zn²⁺, Ni²⁺, Pb²⁺, Cr³⁺ and Cu²⁺, and it shows resistance to lincomycin, norfloxacin, kanamycin, ofloxacin, polymyxin B, ceftriaxone, erythromycin, streptomycin, neomycin, tobramycin, tetracycline and gentamicin, it can be used for the removal of antibiotics and can be used as an excellent bacterial agent of microbial degradation for remediation of antibiotic-contaminated environment, it is especially suitable for the remediation of antibiotic-contaminated environment under heavy metal stress.

In the second aspect of the invention, a bacterial agent is provided, which comprises a heavy metal-antibiotic multiple resistant bacteria described in claim 1.

Preferably, the type of bacterial agent is liquid bacterial agent, powder bacterial agent or granule bacterial agent.

Preferably, the type of bacterial agent is water suspension agent, dispersible oil suspension agent, wettable powder agent or water dispersible granule agent.

Preferably, the bacterial agent also comprises acceptable excipient, the acceptable excipient is selected from one or more of the dispersant, wetting agent, disintegrant, binder, defoaming agent, cryoprotectant, thickener, filler and solvent.

Preferably, the bacterial agent is any of the following (1)-(3).
(1) Bacterial agent for remediation of heavy metal-contaminated water or soil;
(2) Bacterial agent for remediation of antibiotic-contaminated water or soil;
(3) Bacterial agent for remediation of heavy metal and antibiotic-contaminated water or soil.

The heavy metal is one or more of copper ion, zinc ion, cadmium ion, chromium ion, nickel ion or lead ion.

The antibiotic is one or more of lincomycin, norfloxacin, kanamycin, ofloxacin, polymyxin B, ceftriaxone, erythromycin, streptomycin, neomycin, tobramycin, tetracycline or gentamicin.

In the third aspect of the invention, the application of the heavy metal-antibiotic multiple resistant bacteria or bacterial agents in the removal of antibiotics and/or in the preparation of antibiotic degradation agents is provided;

The antibiotics are one or more of lincomycin, norfloxacin, kanamycin, ofloxacin, polymyxin B, ceftriaxone, erythromycin, streptomycin, neomycin, tobramycin, tetracycline or gentamicin.

In the fourth aspect of the invention, the application of the above-mentioned heavy metal-antibiotic multiple resistant bacteria or bacterial agents in the remediation of antibiotic-contaminated environment or heavy metal-contaminated environment or heavy metal and antibiotic-contaminated environment;

The heavy metal is one or more of copper ion, zinc ion, cadmium ion, chromium ion, nickel ion or lead ion.

The antibiotic is one or more of lincomycin, norfloxacin, kanamycin, ofloxacin, polymyxin B, ceftriaxone, erythromycin, streptomycin, neomycin, tobramycin, tetracycline or gentamicin.

In the fifth aspect of the invention, a method for the removal of antibiotics is provided, and the heavy metal-antibiotic multiple resistant bacteria or bacterial agents are directly used for removal treatment in antibiotic-contaminated environment or heavy metal-contaminated environment or heavy metal and antibiotic-contaminated environment.

Preferably, the antibiotic-contaminated environment or heavy metal-contaminated environment or heavy metal and antibiotic-contaminated environment is water or soil contamination.

Especially, the Rossellomorea oryzaecorticis ZC255 strain can be domesticated to improve the removal ability, optimize the removal condition, reduce the substrate concentration of heavy metals and antibiotic pollutants, and add appropriate amount of glucose and other carbon sources to increase the removal rate of Rossellomorea oryzaecorticis ZC255 strain to heavy metals and antibiotic pollutants.

The beneficial effects of the invention are:

The invention provides a heavy metal-antibiotic multiple resistant bacteria, Rossellomorea oryzaecorticis ZC255, the Rossellomorea oryzaecorticis ZC255 strain not only has high activity, simple culture method and strong adaptability, but also can survive in solid medium with high concentration of heavy metals and antibiotics. It has excellent metal tolerance, its maximum tolerance concentration to Cu²⁺ is 7680 mg/L, its maximum tolerance concentration to Cd²⁺ is 820 mg/L, its maximum tolerance concentration to Zn²⁺ is 2660 mg/L, its maximum tolerance concentration to Ni²⁺ is 1210 mg/L, its maximum tolerance concentration to Pb²⁺ is 1640 mg/L, and its maximum tolerance concentration to Cr³⁺ is 400 mg/L. It can effectively degrade lincomycin, norfloxacin, kanamycin, ofloxacin, polymyxin B, ceftriaxone, erythromycin, streptomycin, neomycin, tobramycin, tetracycline, gentamicin, when the initial concentration of lincomycin is 424 mg/L, the absolute adsorption capacity is (32.5±0.4) mg/g, which is suitable for the remediation of antibiotic-contaminated water or soil, especially under heavy metal stress.

The invention utilizes the adsorption removal effect of Rossellomorea oryzaecorticis ZC255 for heavy metals and antibiotics to reduce heavy metals and antibiotics in water or soil, it has the advantages of large adsorption capacity, low cost, high efficiency, good effect and no secondary secondary pollution.

### BRIEF DESCRIPTION OF THE DRAWINGS

The specification drawings that form part of the invention are used to provide a further understanding of the invention, and the schematic embodiments and descriptions of the invention are used to explain the invention, which does not constitute an improper limitation for the invention.

FIG. 1 shows the phylogenetic tree of Rossellomorea oryzaecorticis ZC255.

FIG. 2 shows the result of drug sensitivity test of Rossellomorea oryzaecorticis ZC255.

### DETAILED DESCRIPTION OF THE EMBODIMENTS

Information description of biological preservation materials:

Rossellomorea oryzaecorticis ZC255, taxonomic name: Rossellomorea oryzaecorticis, it has been preserved on Jun. 12,2023 at the China General Microbiological Culture Collection Management Center, the biological preservation number is CGMCC No. 27617, and the address is: No. 3, Courtyard No. 1, Beichen West Road, Chaoyang District, Beijing.

In order to enable the technicians in this field to understand the technical scheme of the invention more clearly, the following will explain the technical scheme of the invention in detail with specific embodiments.

### Embodiment 1: Screening of Rossellomorea oryzaecoricis ZC255 Strain

The Rossellomorea oryzaecorticis ZC255 is isolated and purified from marine sediments of the sea area where is near Shandong Weihai Xiaoshi Island.

### 1. Screening of Strains

Step 1: about 20 g sediment samples are weighed and placed in a 250 mL sterile enrichment bottle, and then the sterile enrichment culture liquid (the enrichment culture liquid comprises aged seawater, 0.2 g/L yeast powder, 0.2 g/tryptone, inorganic salt, 1% (v/v) lincomycin and 2% (v/v) norfloxacin) is added until the bottle is full (about 330 mL), and the lid is tightened to achieve sealing effect to simulate the anoxic environment in the sediment, then the sediment samples are repeatedly inverted and mixed evenly, and conducted enrichment culture at 28° C. for 255 days.
Step 2: the enrichment culture liquid is diluted in a gradient of 101-105, and 200 μL dilution is spread on a solid medium plate and cultured at 30° C. for 48 h.
Step 3: the growth of colony is observed by the naked eye, different forms of single colonies are selected, and streaking is continued to be conduct on a new separation and purification medium plate until the pure bacterial strains with the same colony characteristics are cultured.
Step 4: the isolated pure bacterial strains are inoculated into a solid medium containing copper ions and lincomycin to observe the growth of the strains, and the concentrations of copper and lincomycin are gradually increased, the strains with better growth are selected for preservation, and the strain with the strongest resistance to copper and lincomycin is selected as the target strain, and then it is conducted bacterial strain identification.

### 2. Characteristics of Rossellomorea oryzaecorticis ZC255

### 2.1 Morphological Characteristics.

Step 1: the single colony of the target strain obtained in step 4 is picked up and subjected to three-zone streaking on 2216E solid medium to obtain the single colony.

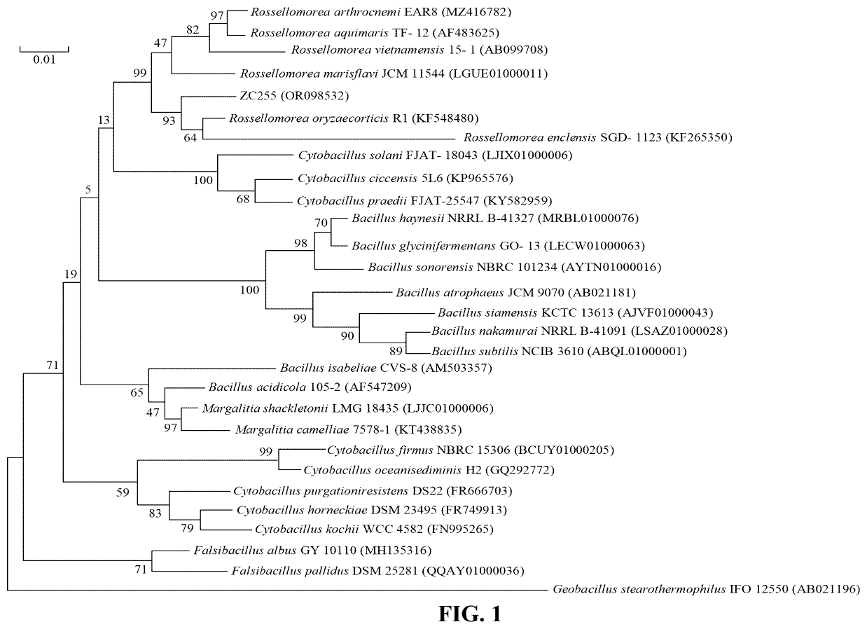

As shown in FIG. 1, the Rossellomorea oryzaecorticis ZC255 colony cultured on 2216E medium at 30° C. for 24 h is observed to be orange-red spherical, Gram staining of this strain shows that it is a Gram-positive bacteria.

2.2 16S rRNA Gene Sequence Analysis.

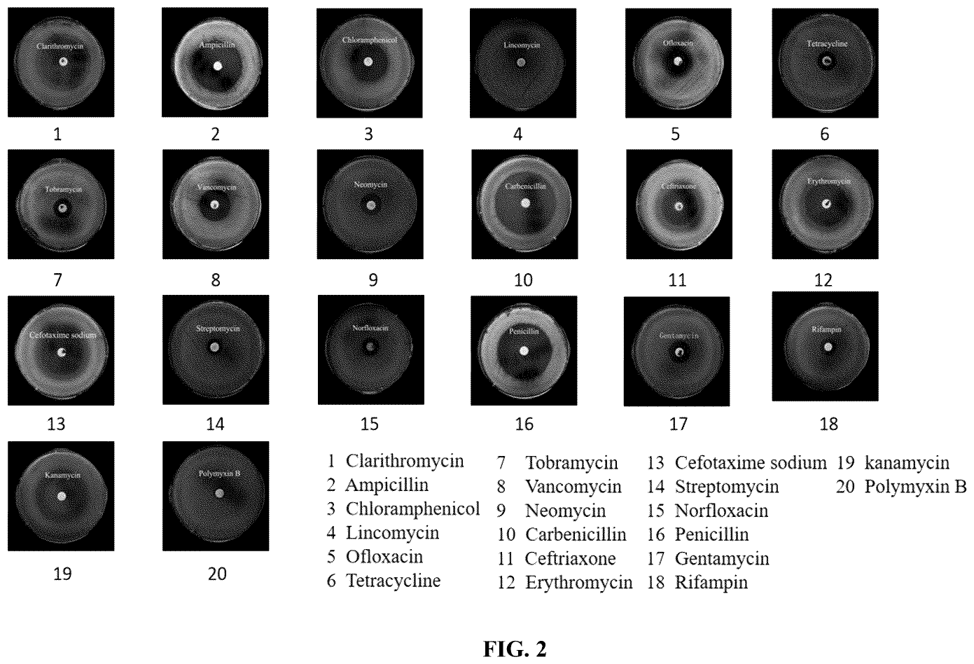

Genomic DNA is extracted from the pure cultured species of Rossellomorea oryzaecorticis ZC255 in Embodiment 1, and the PCR amplification and sequencing are conducted on the pure cultured species, and the phylogenetic tree is constructed by Neighbour-Jioning method using Mega 7.0 software. As shown in FIG. 2, the result shows that the strain belongs to Rossellomorea oryzaecorticis.

### 2.3 Characteristics of Physiological and Biochemical Reaction.

Rossellomorea oryzaecorticis ZC255, the range of pH is 6˜9, the range of temperature is 15˜45° C., and the range of NaCl concentration 0˜18%.

The result of enzyme activity assay shows that Rossellomorea oryzaecorticis ZC255 has oxidase, lipase, caseinase, amylase, cellulase and other activities.

The biochemical characteristics of the strain is tested by using the API reagent Strips and Biolog GEN III MicroPlate. The result of API 20E shows that Citrate utilization, urease, Tryptophan deaminase and Gelatin are positive for ZC255; the result of Biolog GEN III shows that MicroPlate, d-maltose, d-trehalose, d-cellobiose, sucrose, d-turanose, stachyose, positive control, N-acetyl-d-glucosamine, N-acetyl-β-d-mannosamine, N-acetyl-d-galactosamine, N-acetyl neuraminic acid, d-mannose, d-fructose, d-galactose, 3-methyl glucose, d-fucose, L-fucose, L-rhamnose, inosine, d-sorbitol, myo-inositol, pectin, d-gluconic acid, acetoacetic acid and acetic acid tests are also positive for ZC255; and the result of API ZYM shows that 2-Naphthyl octanoate, 2-Naphthyl tetradecanoate, N-Naphthyl-phosphate are positive for ZC255; besides, the result of API 50CH shows that glycol, d-ribose, d-galactose, d-glucose, d-fructose, d-mannose, methyl-αd-glucopyranoside, N-acetylglucosamine, amygdalin, ARBULIN, heptachy botrysum and iron citrate, salicin, d-cellobiose, d-maltose, d-lactose, d-midiose, d-sucrose, d-alginate, d-sonotriose, d-cottonose, starch, glycogen, d-gentian disaccharide, d-turanose, d-tagatose, potassium 5-ketogluconate are positive for ZC255.

### Embodiment 2: Tolerance of Rossellomorea oryzaecorticis ZC255 strain.

### 1. Test

Step 1: the single colonies in Embodiment 1 ‘2.1 morphological characteristics’ are selected and added to the 2216E liquid medium for 24 h to obtain the seed liquid, and then it is added to the liquid containing heavy metals or antibiotics for 48 h.
Step 2: the concentration of heavy metals/antibiotics before and after the addition of seed solution is measured by atomic absorption.

### 2. Test Results

### 2.1 Metal Tolerance.

Rossellomorea oryzaecorticis ZC255 is inoculated into 0, 1600, 3840, 5120, 6400, 7680 and 8192 mg/L of 2216E liquid medium containing Cu2+ (pentahydrate copper sulphate) respectively, the results show that the maximum tolerance concentration of the strain to copper ions is 7680 mg/L, and the growth of the bacteria is not inhibited and the reproduction rate is faster when the concentration of Cu2+ is less than or equal to 1600 mg/L.

In addition, the highest tolerance concentration of the strain to Cd2+is 820 mg/L; the highest tolerance concentration of Zn2+, Ni2+, Pb2+and Cr3+is 2660 mg/L, 1210 mg/L, 1640 mg/L and 400 mg/L respectively.

### 2.2 Antibiotic Tolerance.

Rossellomorea oryzaecorticis ZC255 is tested for drug sensitivity, as shown in Table 1 and FIG. 3, the strain shows drug resistance to lincomycin, norfloxacin, kanamycin, ofloxacin, polymyxin B, ceftriaxone, erythromycin, streptomycin, neomycin, tobramycin, tetracycline and gentamicin.

TABLE 1
Results of drug sensitivity test of strains.

| Antibiotics | Results |
|---|---|
| Lincomycin | R |
| Carbenicillin | S |
| Vancomycin | S |
| Norfloxacin | R |
| Kanamycin | R |
| Ofloxacin | R |
| Ampicillin | S |
| Penicillin | S |
| Polymyxin B | R |
| Ceftriaxone | R |
| Erythromycin | R |
| Chloramphenicol | S |
| Streptomycin | R |
| Clarithromycin | S |
| Rifampicin | I |
| Cefotaxime | I |
| Neomycin | R |
| Tobramycin | R |
| Tetracycline | R |
| Gentamicin | R |

R: resistance, S: sensitivity, I: betweenness.

### 2.2 Adsorption

The Rossellomorea oryzaecorticis ZC255 is inoculated into 2216E liquid medium containing Cu2+ and lincomycin respectively, and it is cultured at 37° C. for 3 days, bacterial liquid is collected and centrifuged at 8000 r/min for 5 min, then the supernatant is harvested. Cu2+ and lincomycin residues are measured to calculate the adsorption capacity.

The biological removal ability of bacterial strains to Cu2+ and lincomycin (Q):

Q=(C0−CL)×V/M

Among them, CL is the final drug concentration (mg/L), CO is the initial drug concentration (mg/L), V is the volume of the culture medium (mL), M is the biomass (g) of Rossellomorea oryzaecorticis ZC255, and Q is the biological removal ability (mg/g) of Rossellomorea oryzaecorticis ZC255. All experiments are in triplicate, and the data are expressed as mean value±standard deviation.

The absolute adsorption capacity of Rossellomorea oryzaecorticis ZC255 strain is (651.4±1.7) mg/g and (350±2.6) mg/g when the initial concentration of copper ion is 3420 mg/L and 1200 mg/L respectively; and the absolute adsorption capacity is (32.5±0.4) mg/g when the initial concentration of lincomycin is 424 mg/L.

In summary, the highest tolerance concentration of Rossellomorea oryzaecorticis ZC255 strain to Cu2+ (7680 mg/L) is significantly higher than that of the reported (350 mg/L), and the biological removal ability of Rossellomorea oryzaecorticis ZC255 strain to Cu2+ (350.2±2.6 mg/g) is significantly higher than that of the reported (73.26±1.61 mg/g).

The above are only the preferred embodiments of the invention, and are not used to limit the invention. For technicians in this field, the invention can have modifications and replacements. Any modifications, equivalent replacements, improvements, etc. made within the spirit and principle of the invention should be included within the protection scope of the invention.

1. A heavy metal-antibiotic multiple resistant bacteria, the heavy metal-antibiotic multiple resistant bacteria is a Rossellomorea oryzaecorticis ZC255, which has been preserved on Jun. 12,2023 at a China General Microbiological Culture Collection Management Center, its biological preservation number is CGMCC No. 27617.

2. A bacterial agent, which comprises the heavy metal-antibiotic multiple resistant bacteria according to claim 1.

3. The bacterial agent according to claim 2, a type of the bacterial agent is a liquid bacterial agent, a powder bacterial agent or a granule bacterial agent.

4. The bacterial agent according to claim 3, the type of the bacterial agent is a water suspension agent, a dispersible oil suspension agent, a wettable powder agent or a water dispersible granule agent.

5. The bacterial agent according to claim 2, the bacterial agent also comprises an acceptable excipient, the acceptable excipient is selected from one or more of a dispersant, a wetting agent, a disintegrant, a binder, a defoaming agent, a cryoprotectant, a thickener, a filler and a solvent.

6. The bacterial agent according to claim 2, the bacterial agent is any of the following (1)-(3):
(1) the bacterial agent for a remediation of a heavy metal-contaminated water or soil;
(2) the bacterial agent for a remediation of an antibiotic-contaminated water or soil;
(3) the bacterial agent for a remediation of a heavy metal and an antibiotic-contaminated water or soil;
a heavy metal is one or more of a copper ion, a zinc ion, a cadmium ion, a chromium ion, a nickel ion or a lead ion; and
an antibiotic is one or more of a lincomycin, a norfloxacin, a kanamycin, an ofloxacin, a polymyxin B, a ceftriaxone, an erythromycin, a streptomycin, a neomycin, a tobramycin, a tetracycline or a gentamicin.

7. An application of the heavy metal-antibiotic multiple resistant bacteria according to claim 1 in a removal of an antibiotic and/or in a preparation of an antibiotic degradation agent;
the antibiotic is one or more of the lincomycin, the norfloxacin, the kanamycin, the ofloxacin, the polymyxin B, the ceftriaxone, the erythromycin, the streptomycin, the neomycin, the tobramycin, the tetracycline or the gentamicin.

8. An application of the heavy metal-antibiotic multiple resistant bacteria according to claim 1 in the remediation of an antibiotic-contaminated environment or a heavy metal-contaminated environment or a heavy metal and antibiotic-contaminated environment;
the heavy metal is one or more of the copper ion, the zinc ion, the cadmium ion, the chromium ion, the nickel ion or the lead ion; and
the antibiotic is one or more of the lincomycin, the norfloxacin, the kanamycin, the ofloxacin, the polymyxin B, the ceftriaxone, the erythromycin, the streptomycin, the neomycin, the tobramycin, the tetracycline or the gentamicin.

9. A method for a removal of an antibiotic, and the heavy metal-antibiotic multiple resistant bacteria according to claim 1 is directly used for a removal treatment in an antibiotic-contaminated environment or a heavy metal-contaminated environment or a heavy metal and antibiotic-contaminated environment.

10. A method for the removal of the antibiotic according to claim 9, the antibiotic-contaminated environment or the heavy metal-contaminated environment or the heavy metal and antibiotic-contaminated environment is a water or soil contamination.

11. An application of the bacterial agent according to claim 2 in a removal of an antibiotic and/or in a preparation of an antibiotic degradation agent;
the antibiotic is one or more of the lincomycin, the norfloxacin, the kanamycin, the ofloxacin, the polymyxin B, the ceftriaxone, the erythromycin, the streptomycin, the neomycin, the tobramycin, the tetracycline or the gentamicin.

12. An application of the bacterial agent according to claim 2 in the remediation of an antibiotic-contaminated environment or a heavy metal-contaminated environment or a heavy metal and antibiotic-contaminated environment;
the heavy metal is one or more of the copper ion, the zinc ion, the cadmium ion, the chromium ion, the nickel ion or the lead ion; and
the antibiotic is one or more of the lincomycin, the norfloxacin, the kanamycin, the ofloxacin, the polymyxin B, the ceftriaxone, the erythromycin, the streptomycin, the neomycin, the tobramycin, the tetracycline or the gentamicin.

13. A method for a removal of an antibiotic, and the bacterial agent according to claim 2 is directly used for a removal treatment in an antibiotic-contaminated environment or a heavy metal-contaminated environment or a heavy metal and antibiotic-contaminated environment.
